(19) United States
(12) Patent Application Publication
(10) Pub. No.: US 2023/0094340 A1
(43) Pub. Date: Mar. 30, 2023

(54) COMPUTING DEVICES PROGRAMMED FOR DYNAMIC ACTIVITY-ASSIGNMENT PROCESSING VIA WEARABLE DEVICES AND METHODS/SYSTEMS OF USE THEREOF

(71) Applicant: StrongArm Technologies, Inc., Brooklyn, NY (US)

(72) Inventors: Michael Patrick Spinelli, Croton, NY (US); SivaSankara Reddy Bommireddy, Secaucus, NJ (US); Jenna Stephenson, Brooklyn, NY (US)

(21) Appl. No.: 17/954,113

(22) Filed: Sep. 27, 2022

Related U.S. Application Data
(60) Provisional application No. 63/249,662, filed on Sep. 29, 2021.

(51) Int. Cl. G16H 20/30; G16H 50/70; A61B 5/11; A61B 5/00; G06T 7/20; G06V 20/52; G06V 40/20; G06V 40/10

(52) U.S. Cl. CPC G16H 20/30; G16H 50/70; A61B 5/1118; A61B 5/4815; A61B 5/681; G06T 7/20; G06V 20/52; G06V 40/23; G06V 40/10; G06T 2207/30196; G06T 2207/30232

(57) ABSTRACT

According to some embodiments, disclosed are systems and methods for dynamic optimized activity-assignment processing based on a periodic assessment of user fatigue risk via wearable devices. The disclosed technology receives tracking data related to movements and activities of a plurality of users working at a location and determines a user-specific injury prone score for each user. Based on this, the technology analyzes a current activity assignment schedule, which is modified to correspond to the current capabilities of each user at the location. As a result, a notification is generated and communicated to each user so as to alert them to their new assignment.

### CROSS-REFERENCE TO RELATED APPLICATIONS

This application claims the benefit of priority from U.S. Provisional Patent Application No. 63/249,662, filed Sep. 29, 2021, which is incorporated herein by reference in its entirety.

### FIELD OF THE DISCLOSURE

The disclosed systems and methods relate to the performance of dynamic optimized activity-assignment processing.

### BACKGROUND

Current techniques for reducing injury and accident related risks are limited to fatigue determination based on sleep and/or by limiting hours worked. However, such solutions fail to account of other factors in fatigue and for acting on the fatigue to reduce accidents and injuries.

### SUMMARY

Some embodiments described herein include a system for performing dynamic optimized activity-assignment processing. In some embodiments, the system includes: a plurality of wearable physical condition tracking devices; and at least one fatigue-based dynamic activity-assignment device; where each of the plurality of wearable physical condition tracking devices is configured to be worn by a user of a plurality of users and to record a user-specific physical condition tracking data during a plurality of time periods while the user performs at least one activity of a plurality of physical activities in at least one physical location of a plurality of physical locations based on a dynamically-updatable activity-assignment data structure; where the user-specific physical condition tracking data includes: a movement-related data; where the at least one fatigue-based dynamic activity-assignment device includes: at least one processor, and a non-transient computer memory, storing fatigue-based dynamic activity-assignment software instructions; where, when the at least one processor executes the fatigue-based dynamic activity-assignment software instructions, for each time period of the plurality of time periods, the at least one processor is programmed to: receive, for each user of the plurality of users, at least: the user-specific physical condition tracking data, and a user-specific activity data; automatically model, for each user of the plurality of users, a user-specific injury-prone fatigue score during each time period based, at least in part, on: the user-specific physical condition tracking data, and the user-specific activity data; automatically utilize an activity-assignment data model to assign the plurality of physical activities across the plurality of users for a subsequent time period to form a subsequent instance of the dynamically-updatable activity-assignment data structure based, at least in part, on: a current instance if the dynamically-updatable activity-assignment data structure, the user-specific injury-prone fatigue score, the user-specific activity data, user-specific activity-specific ability performance data for each physical activity of the plurality of physical activities, activity characteristic data for each physical activity of the plurality of physical activities, and an activity-specific fatigue safety data; transmit a plurality of activity-assignment instructions to a respective plurality of computing devices associated with the plurality of users; and where each activity-assignment instruction of the plurality of activity-assignment instructions direct each user of the plurality of users to perform at least one particular physical activity of the plurality of physical activity during the subsequent time period at the at least one physical location of the plurality of physical locations, based, at least in part, on the subsequent instance of the dynamically-updatable activity-assignment data structure so as to reduce a likelihood of at least one fatigue-caused injury across the plurality of users performing the plurality of physical activities at the plurality of physical locations during the plurality of time periods.

Some embodiments described herein include a method for performing dynamic optimized activity-assignment processing. According to some embodiments, a method is disclosed, which includes: receiving, by a device, for each user of the plurality of users, at least: the user-specific physical condition tracking data, and a user-specific activity data; automatically modelling, by the device, for each user of the plurality of users, a user-specific injury-prone fatigue score during each time period based, at least in part, on: the user-specific physical condition tracking data, and the user-specific activity data; automatically utilizing, by the device, an activity-assignment data model to assign the plurality of physical activities across the plurality of users for a subsequent time period to form a subsequent instance of the dynamically-updatable activity-assignment data structure based, at least in part, on: a current instance if the dynamically-updatable activity-assignment data structure, the user-specific injury-prone fatigue score, user-specific activity data, user-specific activity-specific ability performance data for each physical activity of the plurality of physical activities, activity characteristic data for each physical activity of the plurality of physical activities, and an activity-specific fatigue safety data; and transmitting, by the device, a plurality of activity-assignment instructions to a respective plurality of computing devices associated with the plurality of users, where each activity-assignment instruction of the plurality of activity-assignment instructions direct each user of the plurality of users to perform at least one particular physical activity of the plurality of physical activity during the subsequent time period at the at least one physical location of the plurality of physical locations, based, at least in part, on the subsequent instance of the dynamically-updatable activity-assignment data structure so as to reduce a likelihood of at least one fatigue-caused injury across the plurality of users performing the plurality of physical activities at the plurality of physical locations during the plurality of time periods.

Some of one or more systems and/or methods for performing dynamic optimized activity-assignment processing further include at least one environmental condition tracking device, associated with the at least one physical location of the plurality of physical locations; where the at least one environmental condition tracking device is configured to generate environmental condition data for at least one environmental condition metric; and where, for each time period of the plurality of time periods, the at least one processor is further programmed to: receive, for each user of the plurality of users, the environmental condition data and automatically model, for each user of the plurality of users, the user-specific injury-prone fatigue score during each time period based further on the environmental condition data.

Some of one or more systems and/or methods for performing dynamic optimized activity-assignment processing further include where the at least one environmental condition metric is one of a temperature, a humidity level, or a noise level.

Some of one or more systems and/or methods for performing dynamic optimized activity-assignment processing further include where the activity-assignment data model is a data model associating a plurality of data definitions, including: an activity data definition, identifying each physical activity and a performance complexity of each physical activity so as to form the activity characteristic data, a user activity performance data definition, identifying the at least one physical activity of the plurality of physical activities that each user is capable of performing so as to form the user-specific activity-specific ability performance data, and an activity-specific fatigue safety score data definition, identifying a ranking of the plurality of physical activities among each other from the safest to least safe to be performed when being fatigued so as to form the activity-specific fatigue safety data.

Some of one or more systems and/or methods for performing dynamic optimized activity-assignment processing further include where the activity-specific fatigue safety score data definition is further based, at least in part, on the plurality of physical locations so as to form location-specific activity-specific fatigue safety data; and where, for each time period of the plurality of time periods, the at least one processor is further programmed to automatically utilize the activity-assignment data model to assign the plurality of physical activities across the plurality of users for the subsequent time period to form the subsequent instance of the dynamically-updatable activity-assignment data structure based further on the location-specific activity-specific fatigue safety data.

Some of one or more systems and/or methods for performing dynamic optimized activity-assignment processing further include where the activity-assignment data model is further defined to assign the plurality of physical activities across the plurality of users, by: iteratively identifying, in each iteration, the most fatigue user of the plurality of users; iteratively identifying, in each iteration, a set of physical activities of the plurality of physical activities that the most fatigue user is capable of performing; iteratively identifying, in each iteration, the safest physical activity in the set of physical activities; iteratively checking, in each iteration, for a presence of another user of the plurality of users who is capable of performing a less safe physical activity of the set of physical activities; iteratively assigning, in each iteration, the safest physical activity in the set of physical activities to the most fatigue user when the another user of the plurality of users who is capable of performing the less safe physical activity of the set of physical activities is present; and iteratively assigning, in each iteration, the less safe physical activity in the set of physical activities to the most fatigue user under at least one activity-limiting condition when the another user of the plurality of users who is capable of performing the less safe physical activity of the set of physical activities is not present.

Some of one or more systems and/or methods for performing dynamic optimized activity-assignment processing further include where the at least one activity-limiting condition limits a number of repetitions of the less safe physical activity that the most fatigue user is allowed to perform within the subsequent time period.

Some of one or more systems and/or methods for performing dynamic optimized activity-assignment processing further include where, for each time period of the plurality of time periods, the at least one processor is further programmed to automatically utilize the activity-assignment data model to assign the plurality of physical activities across the plurality of users for the subsequent time period to form the subsequent instance of the dynamically-updatable activity-assignment data structure based further on user-specific historical fatigue data across a set of time periods.

Some of one or more systems and/or methods for performing dynamic optimized activity-assignment processing further include where the set of time periods is equal to or exceeds twenty-four (24) hours.

Some of one or more systems and/or methods for performing dynamic optimized activity-assignment processing further include where each physical activity of the plurality of physical activities is defined based, at least in part, on each job function of a plurality of job functions associated with the plurality of users.

Some of one or more systems and/or methods for performing dynamic optimized activity-assignment processing further include where the plurality of physical locations is within a warehouse.

Some of one or more systems and/or methods for performing dynamic optimized activity-assignment processing further include further including: at least one camera-based tracking device, associated with the at least one physical location of the plurality of physical locations; where the at least one camera-based tracking device is configured to generate visual tracking data for the at least one physical location; and where, for each time period of the plurality of time periods, the at least one processor is further programmed to: receive the visual tracking data, utilize at least one image recognition model to recognize user-specific image data of the at least one user, and automatically model, for each user of the plurality of users, the user-specific injury-prone fatigue score during each time period based further on the user-specific image data.

Some of one or more systems and/or methods for performing dynamic optimized activity-assignment processing further include where the user-specific activity data includes user-specific historical fatigue data representative of past fatigue.

Some of one or more systems and/or methods for performing dynamic optimized activity-assignment processing further include where the user-specific activity data includes user-specific input data, where the user-specific input data includes hours slept data representative of an amount of time that at least one user of the plurality of users slept during a previous night.

Some of one or more systems and/or methods for performing dynamic optimized activity-assignment processing further include where the user-specific input data includes a subjective level of fatigue representative of a user input fatigue level of at least one user of the plurality of users.

### BRIEF DESCRIPTION OF THE DRAWINGS

The foregoing and other objects, features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 illustrates a fatigue evaluation system using a fatigue-based dynamic activity-assignment device 110 for activity assignments in accordance with one or more embodiments of the present disclosure.

FIG. 2 illustrates a feedback cycle implemented by the fatigue-based dynamic activity-assignment device 110 for a user in accordance with one or more embodiments of the present disclosure.

FIG. 3 illustrates a warehouse blueprint showing different levels of risk of the warehouse for use in activity assignment by the activity-assignment data model in accordance with one or more embodiments of the present disclosure. The warehouse blueprint depicts activity area A 301, activity area B 302 and activity area C 303 as assignable physical location for users to perform physical activities. In some embodiments, each of the activity area A 301, activity area B 302 and activity area C 303 may be associated with one or more particular physical activities.

FIG. 4 depicts the logic of the activity-assignment data model in accordance with one or more embodiments of the present disclosure.

FIG. 5 illustrates an alert for an activity-assignment instruction 104 provided to two example users at predetermined time periods in accordance with one or more embodiments of the present disclosure.

FIG. 6 depicts a block diagram of an exemplary computer-based system and platform for fatigue evaluation system in accordance with one or more embodiments of the present disclosure.

FIG. 7 depicts a block diagram of another exemplary computer-based system and platform for fatigue evaluation system in accordance with one or more embodiments of the present disclosure.

FIG. 8 depicts illustrative schematics of an exemplary implementation of the cloud computing/architecture(s) in which embodiments of a system for fatigue evaluation system may be specifically configured to operate in accordance with some embodiments of the present disclosure.

FIG. 9 depicts illustrative schematics of another exemplary implementation of the cloud computing/architecture(s) in which embodiments of a system for fatigue evaluation system may be specifically configured to operate in accordance with some embodiments of the present disclosure.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “runtime” corresponds to any behavior that is dynamically determined during an execution of a software application or at least a portion of software application.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user). The aforementioned examples are, of course, illustrative and not restrictive.

As used herein, the term “user” shall have a meaning of at least one user. In some embodiments, the terms “user”, “subscriber”, “consumer”, or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

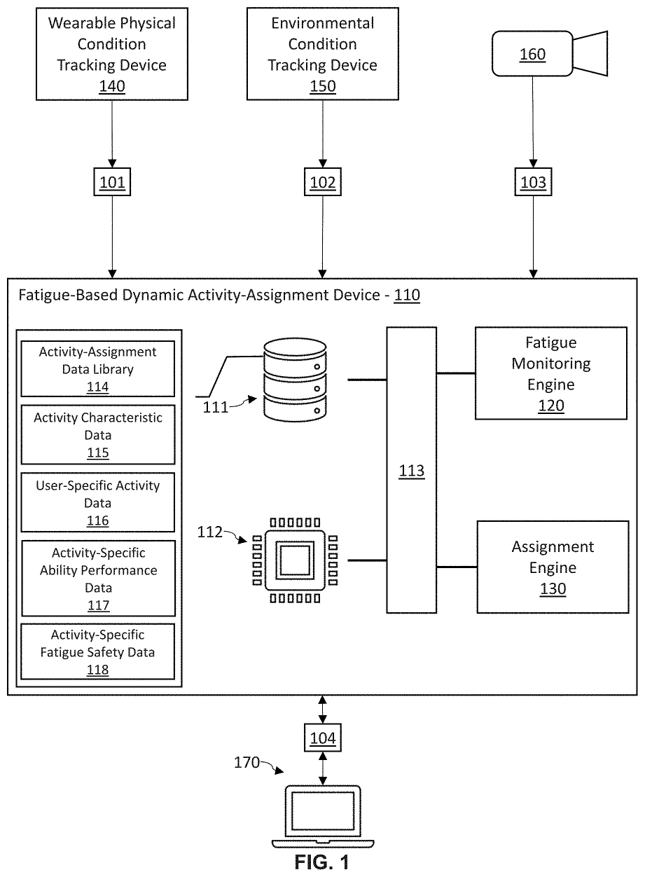

FIG. 1 illustrates a fatigue evaluation system using a fatigue-based dynamic activity-assignment device 110 for activity assignments in accordance with one or more embodiments of the present disclosure.

In some embodiments, a fatigue-based dynamic activity-assignment device 110 calculates a level of fatigue for each industrial athlete based on one or more risk factors. In some embodiments, the level of fatigue may include a fatigue score and/or fatigue level such as, e.g., high, medium and low fatigue, or any other suitable categorization of levels of fatigue on a quantitative and/or qualitative scale, or any combination thereof.

In some embodiments, the fatigue-based dynamic activity-assignment device 110 may calculate the level of fatigue for each industrial athlete on a periodic basis, such as, e.g., every hour, every two hours, every three hours, every four hours, every five hours, every six hours, every seven hours, every eight hours, every nine hours, every ten hours, every eleven hours, every twelve hours, every thirteen hours, every fourteen hours, every fifteen hours, every sixteen hours, every seventeen hours, every nineteen hours, every twenty hours, every twenty-one hours, every twenty two hours, every twenty three hours, every twenty four hours, every two days, every three days, every four days, every five days, every six days, every seven days, every two weeks, every three weeks, every four weeks, every month, or any other suitable period or any suitable combination thereof. In some embodiments, the fatigue evaluation system may calculate the level of fatigue upon a triggering event. In some embodiments, a triggering event may include an industrial athlete signing in to and/or signing out of a time keeping system, a user selection/command to calculate the level of fatigue, an industrial athlete putting on and/or taking off a wearable physical condition tracking device 140 device, or other suitable triggering event.

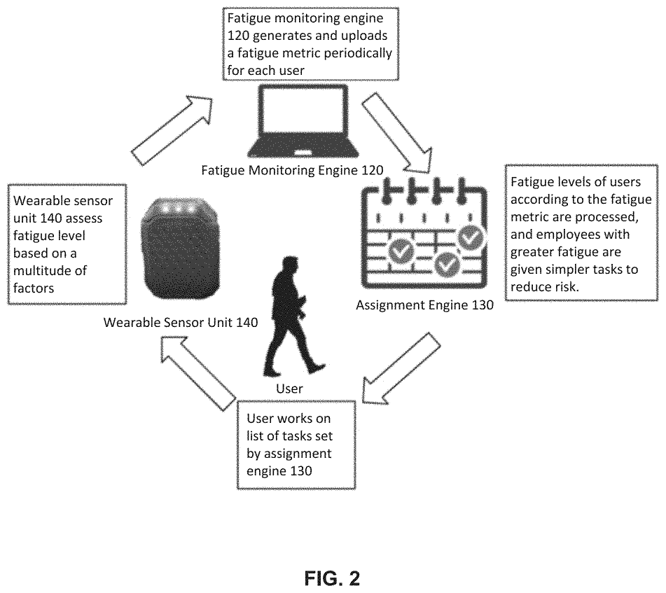

In some embodiments, upon each period of time, the fatigue level is calculated and is wirelessly transmitted to a monitoring system. Based on the level of fatigue of each user a scheduler reassigns existing activities so that the least complex or difficult activities can be assigned to users with higher levels of fatigue. FIG. 2 shows the feedback loop that occurs on a periodic basis. In some embodiments the feedback loop starts with the user wearing the wearable physical condition tracking device 140, a fatigue score being produced and uploaded at a predetermined period of time, and a new set of activities for the next hour determined by the scheduler. Depending on the client, the dynamic scheduler can assign individuals different activities, new frequency expectations of activities (how often an activity should be performed in an hour), sections of the physical locations to work in, or a combination of those.

In some embodiments, the fatigue scores and/or levels are also saved in a data table to allow for aggregate workforce fatigue analysis to be performed. Trends of fatigue over shift times can help clients reassign activities to earlier/later in the shift. In some embodiments, the monitoring system may include components for monitoring user fatigue levels, such as the wearable physical condition tracking device 140 combined with the fatigue monitoring engine 120. As shown in FIG. 1, in some embodiments, the fatigue monitoring engine 120 may be implemented in the fatigue-based dynamic activity-assignment device 110, but in some embodiments may instead be implemented in the wearable device of the wearable physical condition tracking device 140. In some embodiments, the wearable physical condition tracking device 140 may be constantly monitoring the levels of fatigue in each user. In some embodiments, the assignment engine 130 may then reassigning activities every period to reduce risk.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced

Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, the fatigue-based dynamic activity-assignment device 110 may include one or more computer engines implemented on one or more computing devices, server devices, cloud systems, or other suitable device or system or any combination thereof.

In some embodiments, each user may have a wearable physical condition tracking device 140. The wearable physical condition tracking device 140 is configured to be worn by each user for whom fatigue level and assignment recommendations are to be determined. In some embodiments, the wearable physical condition tracking device 140 of each user may record user-specific physical condition tracking data 101 during each time period through time as the user performs one or more physical activities. In some embodiments, each physical activity physical activities may be defined based, at least in part, on one or more activity functions associated with each user. In some embodiments, an activity function may include, e.g., task to be accomplished, movements to be performed, strenuousness or fatigue associated with each movement, physical difficulty, mental difficulty, safety and/or risk, likely severity of a potential injury, among other functions or any suitable combination thereof.

In some embodiments, the user-specific physical condition tracking data 101 may include movement-related data measuring user movements and/or biometric data during movements, as well as a physical location associated with the movement, e.g., within a building, within a factory, within a warehouse, geospatial location, address, or other physical location or any suitable combination thereof.

In some embodiments, the physical activity and/or the user-specific physical condition tracking data 101 may be correlated to one or more physical locations. For example, the physical activity and/or the user may be assigned to particular locations within a facility or to a particular facility or both. In some embodiments, the correlation of the physical location to the physical activity, to the user or both may be recorded in a dynamically-updatable activity-assignment structure. In some embodiments, the dynamically-updatable activity-assignment data structure may record activity assignments that represent an assignment of particular activities to particular users. In some embodiments, the assignments may include particular locations for each particular activity and each particular user. In some embodiments, as the user-specific physical condition tracking data 101 is collected, the fatigue-based dynamic activity-assignment device 110 may determine a fatigue level and dynamically update the dynamically-updatable activity-assignment data structure with recommendations to changes to the assignments of particular activities, particular users and/or particular locations.

In some embodiments, the wearable physical condition tracking device 140 device may include a suitable wearable sensor unit for tracking movement data including, e.g., heart rate, respiration rate, inertial measurement, skin conductance (electrodermal activity (EDA)), body temperature, gait fatigue analysis, sweat detection (skin moisture detection), noise, location (GPS, ultra-wide band) among other user activity, biometric and movement measurements. For example, in some embodiments, the wearable sensor unit may include, e.g., an inertial measurement unit (“IMU”) sensor, the wearable physical condition tracking device 140 records movement data 101 including, e.g., three-dimensional motions of the worker during the day, starting with measurements directly from the three integrated sensors of the IMU. In some embodiments, each sensor reading has an x, y, and z component, yielding a total of nine measurements per data point. In some embodiments, the IMU takes readings from an accelerometer, gyroscope, and magnetometer, each of which measurements has an x, y, and z component. In some embodiments, sensor fusion techniques are applied to filter and integrate the nine-component sensor measurements to calculate the orientation of the single wearable physical condition tracking device 140 mounted to the worker. In some embodiments, the orientation that is calculated in this manner is described by three angles: yaw, pitch, and roll (herein collectively “YPR”). In some embodiments, a sensor fusion algorithm weights the data recorded by the accelerometer, gyroscope, and magnetometer of the IMU to calculate the orientation of the wearable physical condition tracking device 140 in space using quaternion representation. In some embodiments, a sensor fusion algorithm includes a Kalman filter algorithm to process the recorded accelerometer, gyroscope, and magnetometer measurements, to minimize standard sensor noise, and to transform the quaternion representation into yaw, pitch, and roll data.

In some embodiments, the orientation of the wearable physical condition tracking device 140 at any given moment in time can be described by considering an absolute reference frame of three orthogonal axes X, Y, and Z, defined by the Z-axis being parallel and opposite to the Earth's gravity's downward direction, the X-axis pointing towards the Earth's magnetic north, and the Y-axis pointing in a 90-degree counterclockwise rotation from the Z-axis. In some embodiments, the orientation of the wearable physical condition tracking device 140 in space is described as a rotation from the zero-points of this absolute reference frame. In some embodiments, a Tait-Bryan chained rotation (i.e., a subset of Davenport chained rotations) is used to describe the rotation of the wearable physical condition tracking device 140 from the zero points of the absolute reference frame to the orientation of the wearable physical condition tracking device 140 in space. In some embodiments, the rotation is a geometric transformation which takes the yaw, pitch, and roll angles as inputs and outputs a vector that describes the orientation of the wearable physical condition tracking device 140.

In some embodiments, the yaw, pitch, and roll angles that describe the spatial orientation of the wearable physical condition tracking device 140 are used to calculate the yaw, pitch, and roll angles that describe the spatial orientation of the body of the individual to whom the wearable physical condition tracking device 140 is mounted. In some embodiments, to perform this calculation, it is assumed that the wearable physical condition tracking device 140 is rigidly fixed to the initially upright body of the wearer, and the Tait-Bryan chained rotation of the wearable physical condition tracking device 140 is applied in reverse order, to the body, instead of to the wearable physical condition tracking device 140. In some embodiments, the result of this rotation is a vector which can be considered to be the zero point of the body, to which the yaw, pitch, and roll angles of the wearable physical condition tracking device 140 can be applied via a further Tait-Bryan chained rotation to calculate a vector that describes the orientation of the body in space at all times (i.e., a set of YPR values for the body). In some embodiments, parameters that are relevant to the ergonomics of the worker's motions, such as sagittal position, twist position, and lateral position. In some embodiments, the wearable physical condition tracking device 140 is further described in U.S. Pat. No. 10,123,751 attached as Appendix A to this disclosure.

In some embodiments, the fatigue-based dynamic activity-assignment device 110 may receive the user-specific physical condition tracking data 101 for each user from the wearable physical condition tracking device 140 associated with each user. In some embodiments, using the user-specific physical condition tracking data 101, the fatigue-based dynamic activity-assignment device 110 may employ a fatigue monitoring engine 120 to determine a fatigue level for each user, and an assignment engine 130 to determine assignment recommendations and update the dynamically-updatable activity-assignment data structure to provide an activity-assignment instructions 104 to a computing devices 170 associated with the each user in order to effectuate new fatigue-based assignment changes.

In some embodiments, the fatigue-based dynamic activity-assignment device 110 may include one or more computer hardware components such as, e.g., a processor 112, a non-transient computer memory 111, a communication bus 113, among other components or any combination thereof.

In some embodiments, the processor 112 may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processor 112 may include data-processing capacity provided by the microprocessor. In some embodiments, the microprocessor may include memory, processing, interface resources, controllers, and counters. In some embodiments, the microprocessor may also include one or more programs stored in memory.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

In some embodiments, the non-transient computer memory 111 may include, e.g., a suitable memory or storage solutions for maintaining electronic data representing the activity histories for each account. For example, the non-transient computer memory 111 may include database technology such as, e.g., a centralized or distributed database, cloud storage platform, decentralized system, server or server system, among other storage systems. In some embodiments, the non-transient computer memory 111 may, additionally or alternatively, include one or more data storage devices such as, e.g., a hard drive, solid-state drive, flash drive, or other suitable storage device. In some embodiments, the non-transient computer memory 111 may, additionally or alternatively, include one or more temporary storage devices such as, e.g., a random-access memory, cache, buffer, or other suitable memory device, or any other data storage solution and combinations thereof.

In some embodiments, the non-transient computer memory 111 may include, e.g., instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. In some embodiments, the instructions may include instructions for implementing one or models and/or software components of the fatigue monitoring engine 120 and/or the assignment engine 130 such that the processor 112 executes fatigue-based dynamic activity-assignment software instructions, for each time period, to determine a fatigue level and adjustment to the dynamically-updatable activity-assignment data structure to create an assignment adjustment instruction 104.

In some embodiments, the fatigue-based dynamic activity-assignment device 110 may receive the user-specific physical condition tracking data 101 from the wearable physical condition tracking device 140. In some embodiments, the fatigue-based dynamic activity-assignment device 110 may use the user-specific physical condition tracking data 101 with user data and/or activity from the non-transient computer memory 111 to determine a fatigue level and an assignment instruction 104 for each user.

In some embodiments, the user and/or activity data may be accessed via the non-transient computer memory 111 to obtain, e.g., user-specific activity data 116. In some embodiments, the user-specific activity data 116 may include a record of a particular user's activity history, such as, e.g., hours worked in shift, days worked in a row, productivity data, among other user-specific activity data 116. Similarly, the user and/or activity data may include activity-specific ability performance data 117, such as, e.g., data representing the ability of each user to perform each physical activity, including, e.g., a degree to which each user may perform each physical activity, an indication of whether or not each user can perform each physical activity, or a combination thereof.

In some embodiments, the fatigue monitoring engine 120 may communicate with the non-transient computer memory 111 to access the user-specific activity data 116 to determine a fatigue score for a user. In some embodiments, the fatigue monitoring engine 120 may instantiate a fatigue prediction model to automatically model, for each user, a current user-specific injury-prone fatigue score during each time. In some embodiments, the current user-specific injury-prone fatigue score may include a numerical score indicative of a degree of fatigue accumulated through fatigue-inducing factors based on the user-specific activity data 116 and the movement data. In some embodiments, the numerical score may be on a scale from, e.g., 0 to 10, 1 to 10, 0 to 5, 1 to 5, 0 to 20, 1 to 20, or any other suitable scale.

In some embodiments, the fatigue-inducing factors may include the user-specific activity data 116 including, e.g., hours worked in shift; days worked in a row, user-specific historical fatigue data (e.g., past fatigue scores), sleep data (e.g., hours slept the night before), user provided fatigue data (e.g., subjective level of fatigue input by a user), among other user-specific activity data 116. In some embodiments, the fatigue-inducing factors may also include movement data from the user-specific physical condition tracking data 101, including, e.g., gait-related data (e.g., evaluation of gait, steps/day, posture) and/or bend-related data (e.g., count of bends), heart rate fluctuations, respiration rate fluctuations, heart rate variability, skin temperature, skin conductance, distance moved (e.g., using a location device such as GPS and/or UWB), number of steps, among other movement data. In some embodiments, the fatigue level may be determined using one or more aspects of the algorithms described in the documents reproduced as attached in Appendix B.

In some embodiments, the fatigue monitoring engine 120 may include weightings for each fatigue-inducing factor based on the relationship of each fatigue-inducing factor to causing an injury. In some embodiments, the weightings may be predefined, user selected, calculated algorithmically and/or statistically, learned using one or more machine learning models, or any suitable combination thereof.

In some embodiments, the fatigue prediction model may also ingest environmental condition data 102 from one or more environmental condition tracking devices 150 in order to tailor the accumulation of fatigue in each user based on the environmental conditions in which physical activities are performed. In some embodiments, the environmental condition tracking device 150 may include, e.g., sensors on the wearable physical condition tracking device 140, fixed sensor devices associated with particular physical locations, mobile sensor devices stationed in particular physical locations, or any other suitable environmental condition tracking device 150 or any suitable combination thereof.

In some embodiments, the environmental condition data 102 may include, e.g., one or more environmental condition metrics such as, e.g., temperature, humidity, a noise level, air quality, elevation, among other environmental condition metrics or any suitable combination thereof.

In some embodiments, the fatigue monitoring engine 120 may also incorporate video-based tracking of fatigue for more accurate fatigue scoring. In some embodiments, camera-based tracking device(s) 160 may be employed to monitor one or more physical locations during the performance of the physical activities. In some embodiments, the video-based tracking device(s) 160 monitor the physical locations and produce visual tracking data 103, e.g., using thermal imagery, visual imagery, machine vision analysis, etc.). In some embodiments, the video-based tracking data 103 may include, e.g., an indication of the physical activities performed, a duration or number of times associated with the performance of each physical activity, a recognition of users and the physical activities associated with each user, a physical condition (e.g., based on thermal imagery and/or movement analysis) of each user, among other image recognition outputs.

In some embodiments, the fatigue monitoring engine 120 may receive the video-based tracking data 103 and determine a user associated therewith. For example, each camera-based tracking device 160 may capture imagery of one or more users performing one or more physical activities. The camera-based tracking device 160 may identify each user and the physical activities associated with each user, including, e.g., the duration/number of times, physical condition, etc. of each user to produce user-specific video-based tracking data 103. Thus, in some embodiments, the fatigue monitoring engine 120 may use the user-specific video-based tracking data 103 to assess a fatigue level of each user.

In some embodiments, the fatigue-based dynamic activity-assignment device 110 may execute fatigue-based dynamic activity-assignment software instructions to cause the fatigue prediction model of the fatigue monitoring engine 120 may ingest inputs including, e.g., the user-specific activity data 116, the user-specific physical condition tracking data 101, the environmental condition data 102, user-specific video-based tracking data 103, among other suitable inputs or any combination thereof in order to determine a fatigue score for each user. In some embodiments, the fatigue-based dynamic activity-assignment software instructions may be executed periodically for every time period.

In some embodiments, the fatigue prediction model of the fatigue-based dynamic activity-assignment device 110 may process the inputs to produce a user-specific injury-prone fatigue score for a particular time period. The fatigue prediction model performs the analysis every time period with a new batch of input data including, e.g., the user-specific activity data 116, the user-specific physical condition tracking data 101, the environmental condition data 102, among other suitable inputs or any combination thereof. In some embodiments, the new batch of input data may be combined with prior input data. For example, the time period may include, e.g., a one hour period, and the fatigue-based dynamic activity-assignment device 110 may receive new input data every hour, but may use more than one hour worth of input data to make a prediction of a user-specific injury-prone fatigue score for the current one hour period. Thus, the new input data may be combined with old input data to produce a sample period of input data, such as, e.g., two hours, three hours, four hours, five hours, six hours, seven hours, eight hours, time since last waking up, time since last falling asleep, time since last shift, or other suitable sample period.

In some embodiments, the fatigue prediction model of the fatigue-based dynamic activity-assignment device 110 may be configured to utilize one or more exemplary AI/machine learning techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of Neural Network may be executed as follows:
i) define Neural Network architecture/model,
ii) transfer the input data to the exemplary neural network model,
iii) train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In some embodiments, upon predicting the user-specific injury-prone fatigue score for each user, the fatigue monitoring engine 120 may determine a fatigue level of each user, e.g., according to a ranking of fatigue across users (see, Table 1 below). In some embodiments, according to the scale used for the user-specific injury-prone fatigue score, the users may be ranked in order of fatigue score to identify the most to the least fatigued users based on the movement data, environmental condition data 102, and other suitable factors during the current period and/or sampling period.

TABLE 1
A mapping of Fatigue Level to Users for
Fatigue Modelling and Activity Assignment

| | Current |
|---|---|
| Users | Fatigue Score |
| User 1 | Highest |
| User 2 | Lowest |
| User 3 | Medium |

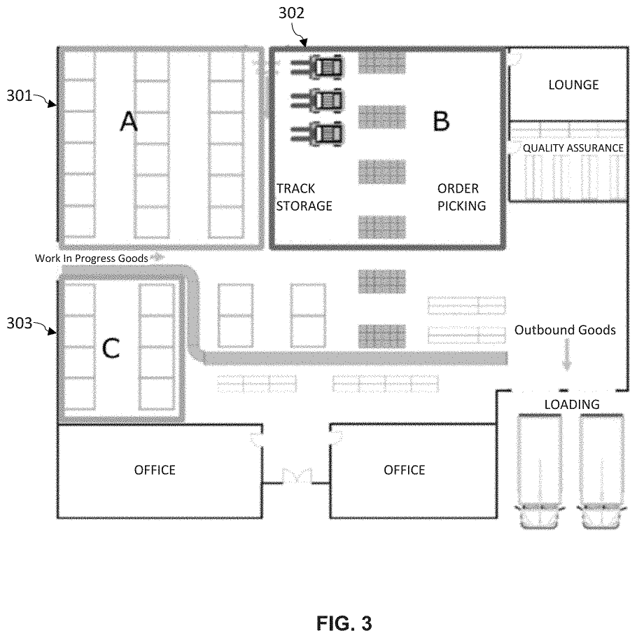

In some embodiments, the user-specific injury-prone fatigue score for each user in a current time period may be provided to the assignment engine 130 to optimizes each user schedule for a subsequent time period (e.g., a next work shift, etc.), taking into account any constraints, such as the ones shown in FIG. 3. In some embodiments, the fatigue monitoring engine 120 may utilize a fatigue prediction model applied to users performing different activities. In some embodiments, the fatigue prediction model may produce a fatigue score and/or fatigue level of each user in order to facilitate the assignment engine 130 to recommend a reassignment of the most complex activities to users with the least amount of fatigue. In some embodiments, the assignment engine 130 may include a constraint that they need to be able to perform that activity.

In some embodiments, the activity-assignment data library 114 may have a current instance of the dynamically-updatable activity-assignment data structure representing current activity assignments to each user. In some embodiments, the assignment engine 130 may access the user-specific injury-prone fatigue score and/or the fatigue level for each user, activity characteristic data 115 and the dynamically-updatable activity-assignment data structure (e.g., in the activity-assignment data library 114 of the non-transient computer memory 111 via the communication but 113) in order to create updated assignments based on the user fatigue levels and characteristics of each physical activity as represented in the activity characteristic data 115. In some embodiments, the activity characteristic data 115 may include, e.g., a complexity, a mental difficulty, a physical difficulty, a risk of consequences (e.g., injury), a severity of potential consequences, among other characteristics of each physical activity. An example of the activity characteristic data 115 is provided in Table 2 below.

TABLE 2
A mapping of Users to Activities based on the complexity of
the activity for Fatigue Modelling and Activity Assignment

| | Complexity | Users that |
|---|---|---|
| | of Activity | can perform |
| Activity | (1-5) | the activity |
| Activity 1 | 5 | User 1 |
| Activity 2 | 3 | User 1, 2, 3 |
| Activity 3 | 1 | User 1, 2 |

Accordingly, in some embodiments, the assignment engine 130 may utilize an activity-assignment data model to balance activity characteristics with user fatigue to minimize the risk of injury while assigning users to the physical activities that they are each able to perform. Thus, the assignment engine 130 may update assignments of the physical activities in the current instance of the dynamically-updatable activity-assignment data structure across each user for a subsequent time period to form a subsequent instance of the dynamically-updatable activity-assignment data structure.

In some embodiments, to do so, the activity-assignment data model may ingest assignment data including, e.g., the current instance if the dynamically-updatable activity-assignment data structure, the user-specific injury-prone fatigue score produced by the fatigue monitoring engine 120, the activity characteristic data 115 for each physical activity, as well as other possible data, including, e.g., the user-specific activity data 116, user-specific activity-specific ability performance data 117 for each physical activity, activity-specific fatigue safety data 118 for each physical activity, user-specific historical fatigue data for each user, among other physical activity and user related data stored in the non-transient computer memory 111 and/or received form external devices, or any combination thereof.

In some embodiments, the activity-assignment data model utilized by the assignment engine 130 is a data model associating data definitions to generate assignments. In some embodiments, the data definitions may include, e.g., an activity data definition identifying each physical activity and a performance complexity of each physical activity so as to form the activity characteristic data 115, a user activity performance data definition identifying the physical activity that each user is capable of performing so as to form the user-specific activity-specific ability performance data 117, and an activity-specific fatigue safety score data definition identifying a ranking of the plurality of physical activities among each other from a safest to a least safe to be performed when being fatigued so as to form the activity-specific fatigue safety data 118.

In some embodiments, the activity-specific fatigue safety score data definition may be based on physical location so as to form location-specific activity-specific fatigue safety data. Some locations in a facility are more injury prone due to, e.g., environmental conditions, dangerous equipment, a need for stairs and/or ladders, among other factors that may increase the probability and/or severity of injury for fatigued users. In some embodiments, for each time period, the processor 112 is further programmed to automatically utilize the activity-assignment data model of the assignment engine 130 to assign the physical activities across each user for the subsequent time period to form the subsequent instance of the dynamically-updatable activity-assignment data structure of the activity-assignment data library 114 based on the location-specific activity-specific fatigue safety data, thus including not only the risks of the physical activities, but also the risks associated with the locations in which each physical activity is performed.

In some embodiments, the activity-assignment data model includes an iterative process for optimizing assignments based on the above described data. In some embodiments, the iterative process may include, e.g.: identifying, in each iteration, a most fatigued user; identifying, in each iteration, a set of physical activities that the most fatigue user is capable of performing; identifying, in each iteration, the safest physical activity in the set of physical activities; checking, in each iteration, for a presence of another user of the plurality of users who is capable of performing a less safe physical activity of the set of physical activities; assigning, in each iteration, the safest physical activity in the set of physical activities to the most fatigued user when another user of the plurality of users who is capable of performing the less safe physical activity of the set of physical activities is present; and assigning, in each iteration, the less safe physical activity in the set of physical activities to the most fatigued user under an activity-limiting condition when another user who is capable of performing the less safe physical activity of the set of physical activities is not present. Accordingly, in some embodiments, the activity-assignment data model may iteratively reassign for each physical activity and each user the most fatigued users to minimize the risk of injury. Thus, the most fatigued users are moved to safer jobs to compensate for the fatigue, which would otherwise increase the risk of injury.

In some embodiments, an example of the activity-limiting condition includes a limit to a number of repetitions of the less safe physical activity that the most fatigued user is allowed to perform within the subsequent time period.

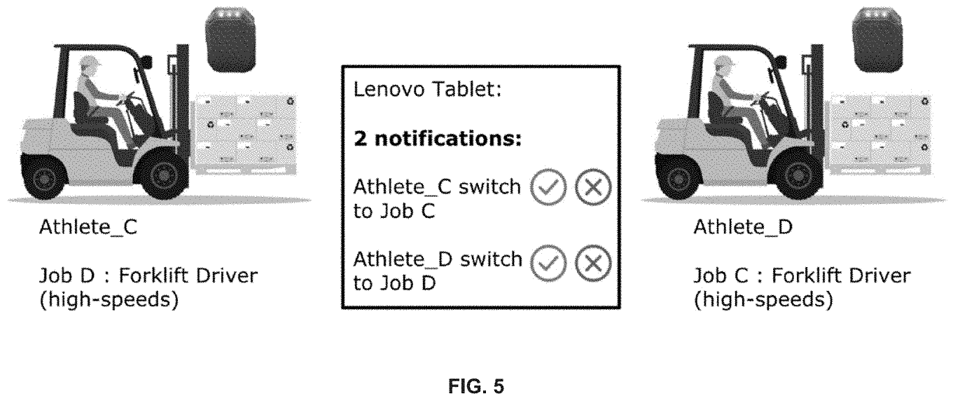

In some embodiments, upon producing the subsequent dynamically-updatable activity-assignment data structure, the assignment engine 130 may generate one or more activity-assignment instructions 104. In some embodiments, an activity-assignment instruction 104 may include an instruction to a user regarding an assignment to a physical activity for the subsequent time period (see, for example, FIG. 5 below). In some embodiments, each activity-assignment instruction 104 may be provided to a computing device 170 where a user can view and act on the activity-assignment instruction 104. In some embodiments, the computing device 170 may include a user device associated with a particular user, such as, e.g., a mobile device (e.g., a mobile phone, Personal Digital Assistant (PDA), Blackberry™, Pager, Smartphone, tablet, smart glasses, augmented reality (AR) headset, virtual reality (VR) headset, smart watch, or any other reasonable mobile electronic device), a laptop computing device, desktop computing device, terminal device, thin client, or any other suitable computing device 170. In some embodiments, the computing device 170 may include a shared computing device accessible by multiple users, such as, e.g., computing device associated with a particular location, department, organization, etc. In some embodiments, each activity-assignment instruction 104 directs a user to perform at least one particular physical activity during the subsequent time period at the physical location, based, at least in part, on the subsequent instance of the dynamically-updatable activity-assignment data structure so as to reduce a likelihood of at least one fatigue-caused injury across the users performing the physical activities at the physical locations during the subsequent time period.

FIG. 2 illustrates a feedback cycle occurring for an athlete during the day. The individual wears a wearable physical condition tracking device 140, the wearable physical condition tracking device 140 produces a fatigue score that is uploaded hourly to the monitoring system, and the scheduling system reassigns work based on fatigue level.

FIG. 3 illustrates a warehouse blueprint showing different levels of risk of the warehouse. The example schedules show a first and a second hour of work to work zones of FIG. 1. In some embodiments, the second schedule (Table 4) shows changes to assigned work zones relative to a first schedule (Table 3) after a relative fatigue analysis is uploaded from the first period of work. Each period, the schedule updates according to the newest fatigue level based on the fatigue monitoring engine 120 and the assignment engine 130 as described above.

TABLE 3
Assigned Work Zone by user during
a first shift period (Shift Hour 0)

| Shift | | Current | Assigned |
|---|---|---|---|
| Hour | Name | Fatigue Score | Work Zone |
| 0 | User-1 | N/A | A |
| 0 | User-2 | N/A | B |
| 0 | User-3 | N/A | C |

TABLE 4
Assigned Work Zone by user during a second shift period
(Shift Hour 1) based on a Current Fatigue Score

| Shift | | Current | Assigned |
|---|---|---|---|
| Hour | Name | Fatigue Score | Work Zone |
| 1 | User-1 | Highest | C |
| 1 | User-2 | Lowest | B |
| 1 | User-3 | Medium | A |

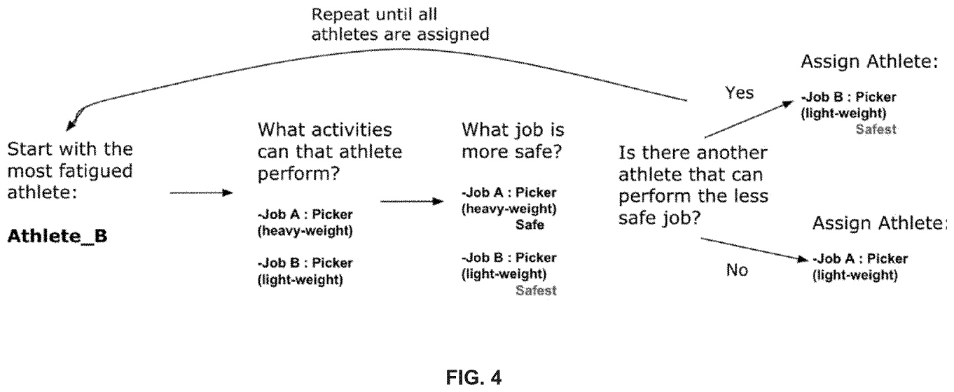

FIG. 4 depicts the logic of the activity-assignment data model. In some embodiments, the logic pairs the most fatigued user with the safest activity, unless no one else can perform the activity.

FIG. 5 illustrates an alert for an activity-assignment instruction 104 provided TO two example users at predetermined time periods. In some embodiments, the users may access the computing device 170 to view activity assignments and/or any changes to previous activity assignments. In some embodiments, the example depicted in FIG. 5, each user may select a check mark to acknowledge the assignment and remove the notification.

In some embodiments, the activity-specific fatigue safety data 118 may include data for quantitatively assessing safety and risk factors, such as, e.g., relative mental difficulty, relative physical difficulty, potential consequences, potential severity of injury, potential risk of injury, potential risk of severe injury and potential risk of minor injury, among other factors or any combination thereof. In some embodiments, each safety and risk factor may be, e.g., predefined, user selectable, learned via a suitable machine learning model (e.g., linear regression, a neural network, clustering, etc.), or according to any other suitable technique or any combination thereof.

In some embodiments, the safety and risk factors may be aggregated to form fatigue safety ranking for each activity type. In some embodiments, the safety and risk factors may be aggregated according to any suitable aggregation methodology, such as, e.g., a sum, a product, an average, a weighted sum, a weighted product, a weighted average, a regression, among others or any suitable combination thereof.

Table 5 and 6, and FIGS. 4 and 5 provide a detailed example of the dynamically-updatable activity-assignment data structure and its use to recommend activity assignments based on fatigue level. Table 5 shows two data-frames, one with the information about activity complexity, and the other with an hourly “schedule update” showing a fatigue level of each user and any changes in the activity assigned to each user. FIG. 4 shows the logic that happens behind the algorithm and FIG. 5 demonstrates what the athletes see from their end.

In some embodiments, fatigue-based dynamic activity-assignment device 110, therefore, solves the problem of reducing risk by taking fatigue into consideration and producing actionable assignment recommendations. When fatigue levels are high in an individual, they are assigned to a less dangerous part of the warehouse or given activities that are less complex. The outcome is a reduction in injuries.

TABLE 5
Example activity-specific fatigue safety data 118 for four example activities.

| | | Users able | Relative | Relative | | Fatigue Safety |
|---|---|---|---|---|---|---|
| Type of | | to perform | Mental | Physical | Potential | Ranking (lowest |
| Activity | Description | activity | Difficulty | Difficulty | Consequences | is least safe) |
| Activity A: | This worker | User_A, | 4 | 1 | 3 | 8: Safe |
| Picker | listens to his | User_B, | | | | |
| (heavy- | headset and | User_C | | | | |
| weight) | picks up items | | | | | |
| | to place on a | | | | | |
| | pallet. This | | | | | |
| | activity is for | | | | | |
| | articles <0 lbs | | | | | |
| Activity B: | This worker | User_A, | 3 | 2 | 4 | 9: Safest |
| Picker | listens to his | User_B, | | | | |
| (light- | headset and | User_C, | | | | |
| weight) | picks up items | User_D | | | | |
| | lo place on a | | | | | |
| | pallet. This | | | | | |
| | activity is for | | | | | |
| | articles <20 lbs. | | | | | |
| Activity C: | Forklift drivers | User_C, | 2 | 3 | 2 | 7: Medium Safe |
| Forklift | pick heavy items | User_D | | | | |
| Driver | and transport | | | | | |
| (low- | them to another | | | | | |
| speeds) | area. This | | | | | |
| | activity doesn't | | | | | |
| | allow the | | | | | |
| | forklift to | | | | | |
| | exceed 5 mph. | | | | | |
| Activity D: | Forklift drivers | User_C, | 1 | 3 | 1 | 5: Least Safe |
| Forklift | pick heavy | User_D | | | | |
| Driver | items and | | | | | |
| (high- | transport them | | | | | |
| speeds) | to another area. | | | | | |
| | This activity | | | | | |
| | allows for any | | | | | |
| | forklift speed. | | | | | |

In some embodiments, the output recommends a switch of activity function of a more dangerous activity from a first user to a second user that is less fatigued. For example, Table 6 below shows an example where User_C and User_D are recommended to switch assignments since User_D is less fatigued than User_C, and Activity D is higher risk than Activity C. In some embodiments, even though User_C is more fatigued than User_A, User_A is not switched to Activity C because User_A does not have the training to perform that activity.

TABLE 6
An example of a 9:00 AM assignment based on the periodic fatigue analysis.

| | | Gait | Bend | | Location | | | Next |
|---|---|---|---|---|---|---|---|---|
| | | Analysis | Count (>60 | Hours | Safety | | Fatigue | Activity |
| User | Timestamp | Fatigue (1-10) | Degrees) | Worked | (1-10) | Current Activity | Score | Assigned |
| User_A | 2021-09-15, | 3 | 75 | 2 | 1 | Activity A: | Medium | Activity A: |
| | 09:00:00 | | | | | Picker | fatigue | Picker |
| | | | | | | (heavy-weight) | | (heavy-weight) |
| User_B | 2021-09-15, | 5 | 130 | 6 | 1 | Activity B: | Most | Activity B: |
| | 09:00:00 | | | | | Picker | fatigue | Picker |
| | | | | | | (light-weight) | | (light-weight) |
| User_C | 2021-09-15, | 0 | 5 | 4 | 6 | Activity D: | Fatigue | Activity C: |
| | 09:00:00 | | | | | Forklift Driver | | Forklift Driver |
| | | | | | | (high-speeds) | | (low-speeds) |
| User_D | 2021-09-15, | 0 | 7 | 1 | 6 | Activity C: | Least | Activity D: |
| | 09:00:00 | | | | | Forklift Driver | fatigue | Forklift Driver |
| | | | | | | (low-speeds) | | (high-speeds) |

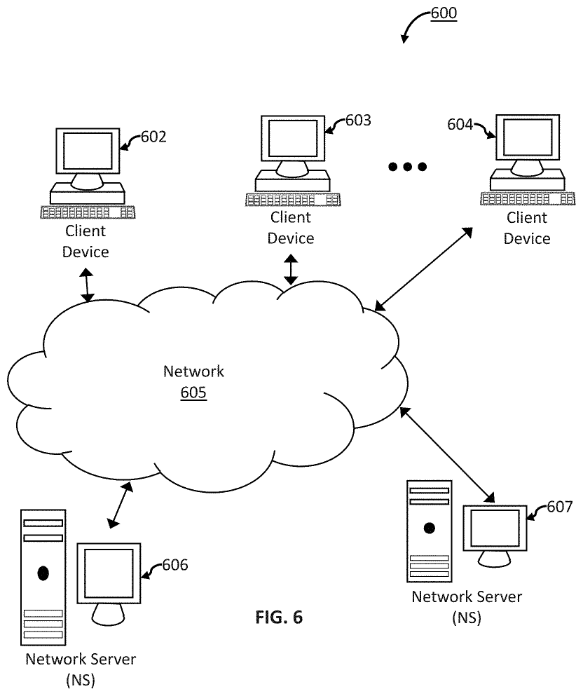

FIG. 6 depicts a block diagram of an exemplary computer-based system and platform 600 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 600 may be configured to manage a large number of members and concurrent transactions, as detailed herein. In some embodiments, the exemplary computer-based system and platform 600 may be based on a scalable computer and network architecture that incorporates varies strategies for assessing the data, caching, searching, and/or database connection pooling. An example of the scalable architecture is an architecture that is capable of operating multiple servers.

In some embodiments, referring to FIG. 6, member computing device 602, member computing device 603 through member computing device 604 (e.g., clients) of the exemplary computer-based system and platform 600 may include virtually any computing device capable of receiving and sending a message over a network (e.g., cloud network), such as network 605, to and from another computing device, such as servers 606 and 607, each other, and the like. In some embodiments, the member devices 602-604 may be personal computers, multiprocessor systems, microprocessor-based or programmable consumer electronics, network PCs, and the like. In some embodiments, one or more member devices within member devices 602-604 may include computing devices that typically connect using a wireless communications medium such as cell phones, smart phones, pagers, walkie talkies, radio frequency (RF) devices, infrared (IR) devices, citizens band radio, integrated devices combining one or more of the preceding devices, or virtually any mobile computing device, and the like. In some embodiments, one or more member devices within member devices 602-604 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, OFDM, OFDMA, LTE, satellite, ZigBee, etc.). In some embodiments, one or more member devices within member devices 602-604 may include may run one or more applications, such as Internet browsers, mobile applications, voice calls, video games, videoconferencing, and email, among others. In some embodiments, one or more member devices within member devices 602-604 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a member device within member devices 602-604 may be specifically programmed by either Java, .Net, QT, C, C++, Python, PHP and/or other suitable programming language. In some embodiment of the device software, device control may be distributed between multiple standalone applications. In some embodiments, software components/applications can be updated and redeployed remotely as individual units or as a full software suite. In some embodiments, a member device may periodically report status or send alerts over text or email. In some embodiments, a member device may contain a data recorder which is remotely downloadable by the user using network protocols such as FTP, SSH, or other file transfer mechanisms. In some embodiments, a member device may provide several levels of user interface, for example, advance user, standard user. In some embodiments, one or more member devices within member devices 602-604 may be specifically programmed include or execute an application to perform a variety of possible tasks, such as, without limitation, messaging functionality, browsing, searching, playing, streaming or displaying various forms of content, including locally stored or uploaded messages, images and/or video, and/or games.

In some embodiments, the exemplary network 605 may provide network access, data transport and/or other services to any computing device coupled to it. In some embodiments, the exemplary network 605 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the exemplary network 605 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 605 may include and implement, as an alternative or in conjunction with one or more of the above, a WiMAX architecture defined by the WiMAX forum. In some embodiments and, optionally, in combination of any embodiment described above or below, the exemplary network 605 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 605 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, OFDM, OFDMA, LTE, satellite and any combination thereof. In some embodiments, the exemplary network 605 may also include mass storage, such as network attached storage (NAS), a storage area network (SAN), a content delivery network (CDN) or other forms of computer or machine readable media.

In some embodiments, the exemplary server 606 or the exemplary server 607 may be a web server (or a series of servers) running a network operating system, examples of which may include but are not limited to Apache on Linux or Microsoft IIS (Internet Information Services). In some embodiments, the exemplary server 606 or the exemplary server 607 may be used for and/or provide cloud and/or network computing. Although not shown in FIG. 6, in some embodiments, the exemplary server 606 or the exemplary server 607 may have connections to external systems like email, SMS messaging, text messaging, ad content providers, etc. Any of the features of the exemplary server 606 may be also implemented in the exemplary server 607 and vice versa.

In some embodiments, one or more of the exemplary servers 606 and 607 may be specifically programmed to perform, in non-limiting example, as authentication servers, search servers, email servers, social networking services servers, Short Message Service (SMS) servers, Instant Messaging (IM) servers, Multimedia Messaging Service (MMS) servers, exchange servers, photo-sharing services servers, advertisement providing servers, financial/banking-related services servers, travel services servers, or any similarly suitable service-base servers for users of the member computing devices 601-604.

In some embodiments and, optionally, in combination of any embodiment described above or below, for example, one or more exemplary computing member devices 602-604, the exemplary server 606, and/or the exemplary server 607 may include a specifically programmed software module that may be configured to send, process, and receive information using a scripting language, a remote procedure call, an email, a tweet, Short Message Service (SMS), Multimedia Message Service (MMS), instant messaging (IM), an application programming interface, Simple Object Access Protocol (SOAP) methods, Common Object Request Broker Architecture (CORBA), HTTP (Hypertext Transfer Protocol), REST (Representational State Transfer), SOAP (Simple Object Transfer Protocol), MLLP (Minimum Lower Layer Protocol), or any combination thereof.

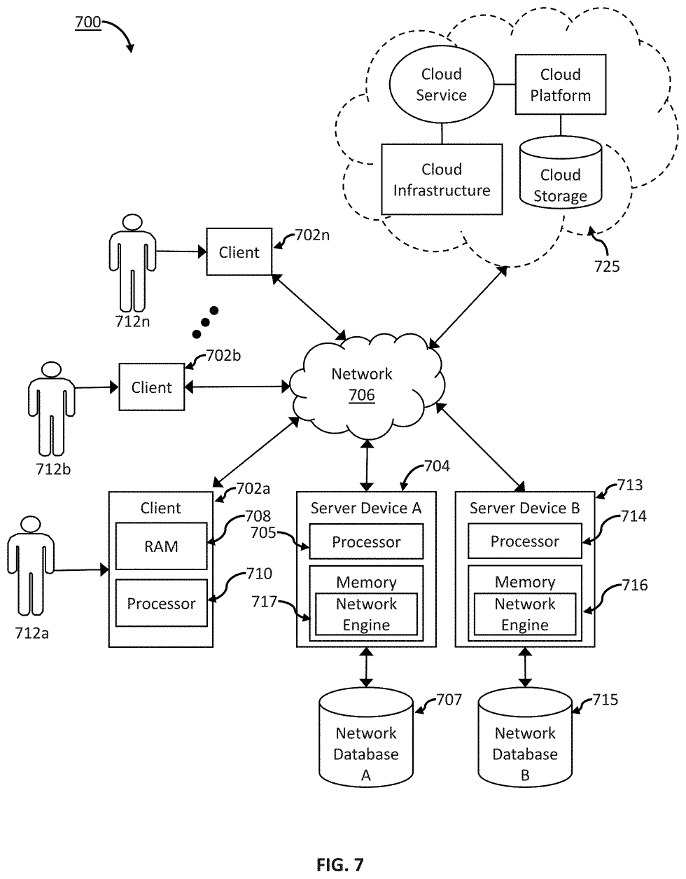

FIG. 7 depicts a block diagram of another exemplary computer-based system and platform 700 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the member computing devices 702a, 702b thru 702n shown each at least includes a computer-readable medium, such as a random-access memory (RAM) 708 coupled to a processor 710 or FLASH memory. In some embodiments, the processor 710 may execute computer-executable program instructions stored in memory 708. In some embodiments, the processor 710 may include a microprocessor, an ASIC, and/or a state machine. In some embodiments, the processor 710 may include, or may be in communication with, media, for example computer-readable media, which stores instructions that, when executed by the processor 710, may cause the processor 710 to perform one or more steps described herein. In some embodiments, examples of computer-readable media may include, but are not limited to, an electronic, optical, magnetic, or other storage or transmission device capable of providing a processor, such as the processor 710 of client 702a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, and etc.

In some embodiments, member computing devices 702a through 702n may also comprise a number of external or internal devices such as a mouse, a CD-ROM, DVD, a physical or virtual keyboard, a display, or other input or output devices. In some embodiments, examples of member computing devices 702a through 702n (e.g., clients) may be any type of processor-based platforms that are connected to a network 706 such as, without limitation, personal computers, digital assistants, personal digital assistants, smart phones, pagers, digital tablets, laptop computers, Internet appliances, and other processor-based devices. In some embodiments, member computing devices 702a through 702n may be specifically programmed with one or more application programs in accordance with one or more principles/methodologies detailed herein. In some embodiments, member computing devices 702a through 702n may operate on any operating system capable of supporting a browser or browser-enabled application, such as Microsoft™ Windows™, and/or Linux. In some embodiments, member computing devices 702a through 702n shown may include, for example, personal computers executing a browser application program such as Microsoft Corporation's Internet Explorer™, Apple Computer, Inc.'s Safari™, Mozilla Firefox, and/or Opera. In some embodiments, through the member computing client devices 702a through 702n, users, 712a through 702n, may communicate over the exemplary network 706 with each other and/or with other systems and/or devices coupled to the network 706. As shown in FIG. 7, exemplary server devices 704 and 713 may include processor 705 and processor 714, respectively, as well as memory 717 and memory 716, respectively. In some embodiments, the server devices 704 and 713 may be also coupled to the network 706. In some embodiments, one or more member computing devices 702a through 702n may be mobile clients.

In some embodiments, at least one database of exemplary databases 707 and 715 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

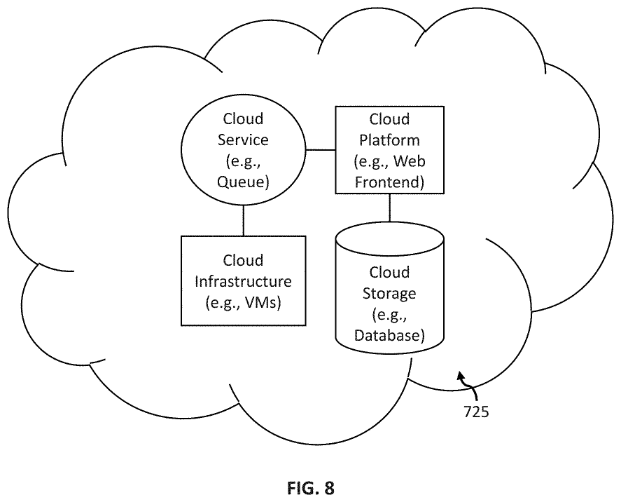

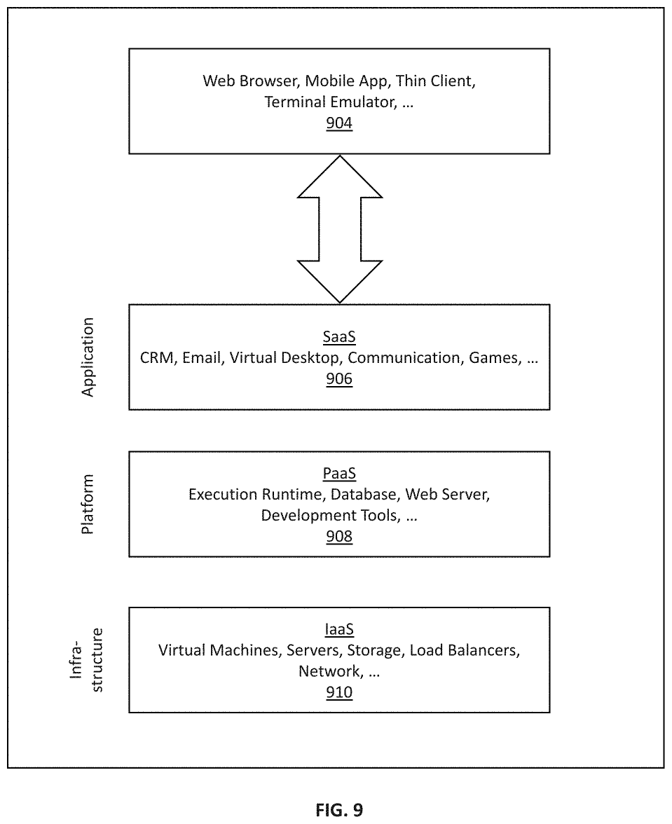

In some embodiments, the exemplary inventive computer-based systems/platforms, the exemplary inventive computer-based devices, and/or the exemplary inventive computer-based components of the present disclosure may be specifically configured to operate in a cloud computing/architecture 725 such as, but not limiting to: infrastructure a service (IaaS) 910, platform as a service (PaaS) 908, and/or software as a service (SaaS) 906 using a web browser, mobile app, thin client, terminal emulator or other endpoint 904. FIGS. 8 and 9 illustrate schematics of exemplary implementations of the cloud computing/architecture(s) in which the exemplary systems of the present disclosure may be specifically configured to operate.

The aforementioned examples are, of course, illustrative and not restrictive.

At least some aspects of the present disclosure will now be described with reference to the following numbered clauses.
1. A system including:
   a plurality of wearable physical condition tracking devices; and
   at least one fatigue-based dynamic activity-assignment device;
   where each of the plurality of wearable physical condition tracking devices is configured to be worn by a user of a plurality of users and to record a user-specific physical condition tracking data during a plurality of time periods while the user performs at least one activity of a plurality of physical activities in at least one physical location of a plurality of physical locations based on a dynamically-updatable activity-assignment data structure;
   where the user-specific physical condition tracking data includes:
      a movement-related data;
   where the at least one fatigue-based dynamic activity-assignment device includes:
      at least one processor, and
      a non-transient computer memory, storing fatigue-based dynamic activity-assignment software instructions;
      where, when the at least one processor executes the fatigue-based dynamic activity-assignment software instructions, for each time period of the plurality of time periods, the at least one processor is programmed to:
         receive, for each user of the plurality of users, at least:
            the user-specific physical condition tracking data and
            a user-specific activity data;
            automatically model, for each user of the plurality of users, a user-specific injury-prone fatigue score during each time period based, at least in part, on:
            the user-specific physical condition tracking data and
            the user-specific activity data;
            automatically utilize an activity-assignment data model to assign the plurality of physical activities across the plurality of users for a subsequent time period to form a subsequent instance of the dynamically-updatable activity-assignment data structure based, at least in part, on:
            a current instance if the dynamically-updatable activity-assignment data structure,
            the user-specific injury-prone fatigue score,
            the user-specific activity data,
            user-specific activity-specific ability performance data for each physical activity of the plurality of physical activities,
            activity characteristic data for each physical activity of the plurality of physical activities, and
            an activity-specific fatigue safety data;
            transmit a plurality of activity-assignment instructions to a respective plurality of computing devices associated with the plurality of users; and
            where each activity-assignment instruction of the plurality of activity-assignment instructions direct each user of the plurality of users to perform at least one particular physical activity of the plurality of physical activity during the subsequent time period at the at least one physical location of the plurality of physical locations, based, at least in part, on the subsequent instance of the dynamically-updatable activity-assignment data structure so as to reduce a likelihood of at least one fatigue-caused injury across the plurality of users performing the plurality of physical activities at the plurality of physical locations during the plurality of time periods.
2. The system of Clause 1, further including:
   at least one environmental condition tracking device, associated with the at least one physical location of the plurality of physical locations;
   where the at least one environmental condition tracking device is configured to generate environmental condition data for at least one environmental condition metric; and
   where, for each time period of the plurality of time periods, the at least one processor is further programmed to:
      receive, for each user of the plurality of users, the environmental condition data and
      automatically model, for each user of the plurality of users, the user-specific injury-prone fatigue score during each time period based further on the environmental condition data.
3. The system of Clause 2, where the at least one environmental condition metric is one of a temperature, a humidity level, or a noise level.
4. The system of Clause 1, where the activity-assignment data model is a data model associating a plurality of data definitions, including:
   an activity data definition, identifying each physical activity and a performance complexity of each physical activity so as to form the activity characteristic data,
   a user activity performance data definition, identifying the at least one physical activity of the plurality of physical activities that each user is capable of performing so as to form the user-specific activity-specific ability performance data, and
   an activity-specific fatigue safety score data definition, identifying a ranking of the plurality of physical activities among each other from the safest to least safe to be performed when being fatigued so as to form the activity-specific fatigue safety data.
5. The system of Clause 4,
   where the activity-specific fatigue safety score data definition is further based, at least in part, on the plurality of physical locations so as to form location-specific activity-specific fatigue safety data; and
   where, for each time period of the plurality of time periods, the at least one processor is further programmed to automatically utilize the activity-assignment data model to assign the plurality of physical activities across the plurality of users for the subsequent time period to form the subsequent instance of the dynamically-updatable activity-assignment data structure based further on the location-specific activity-specific fatigue safety data.
6. The system of Clause 4, where the activity-assignment data model is further defined to assign the plurality of physical activities across the plurality of users, by:
   iteratively identifying, in each iteration, the most fatigue user of the plurality of users;
   iteratively identifying, in each iteration, a set of physical activities of the plurality of physical activities that the most fatigue user is capable of performing;
   iteratively identifying, in each iteration, the safest physical activity in the set of physical activities;
   iteratively checking, in each iteration, for a presence of another user of the plurality of users who is capable of performing a less safe physical activity of the set of physical activities;
   iteratively assigning, in each iteration, the safest physical activity in the set of physical activities to the most fatigue user when the other user of the plurality of users who is capable of performing the less safe physical activity of the set of physical activities is present; and
   iteratively assigning, in each iteration, the less safe physical activity in the set of physical activities to the most fatigue user under at least one activity-limiting condition when the other user of the plurality of users who is capable of performing the less safe physical activity of the set of physical activities is not present.
7. The system of Clause 6, where the at least one activity-limiting condition limits a number of repetitions of the less safe physical activity that the most fatigue user is allowed to perform within the subsequent time period.
8. The system of Clause 1, where, for each time period of the plurality of time periods, the at least one processor is further programmed to automatically utilize the activity-assignment data model to assign the plurality of physical activities across the plurality of users for the subsequent time period to form the subsequent instance of the dynamically-updatable activity-assignment data structure based further on user-specific historical fatigue data across a set of time periods.
9. The system of Clause 8, where the set of time periods is equal to or exceeds twenty-four (24) hours.
10. The system of Clause 1, where each physical activity of the plurality of physical activities is defined based, at least in part, on each job function of a plurality of job functions associated with the plurality of users.
11. The system of Clause 1, where the plurality of physical locations is within a warehouse.
12. The system of Clause 1, further including:
   at least one camera-based tracking device, associated with the at least one physical location of the plurality of physical locations;
   where the at least one camera-based tracking device is configured to generate visual tracking data for the at least one physical location; and
   where, for each time period of the plurality of time periods, the at least one processor is further programmed to:
      receive the visual tracking data,
      utilize at least one image recognition model to recognize user-specific image data of the at least one user, and
      automatically model, for each user of the plurality of users, the user-specific injury-prone fatigue score during each time period based further on the user-specific image data.
13. The system of Clause 1, where the user-specific activity data includes user-specific historical fatigue data representative of past fatigue.
14. The system of Clause 1, where the user-specific activity data includes user-specific input data.
15. The system of Clause 14, where the user-specific input data includes hours slept data representative of an amount of time that at least one user of the plurality of users slept during a previous night.
16. The system of Clause 1, where the user-specific input data includes a subjective level of fatigue representative of a user input fatigue level of at least one user of the plurality of users.

While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

What is claimed is:

1. A system including:
a plurality of wearable physical condition tracking devices; and
at least one fatigue-based dynamic activity-assignment device;
where each of the plurality of wearable physical condition tracking devices is configured to be worn by a user of a plurality of users and to record a user-specific physical condition tracking data during a plurality of time periods while the user performs at least one activity of a plurality of physical activities in at least one physical location of a plurality of physical locations based on a dynamically-updatable activity-assignment data structure;
where the user-specific physical condition tracking data includes:
a movement-related data;
where the at least one fatigue-based dynamic activity-assignment device includes:
at least one processor, and
a non-transient computer memory, storing fatigue-based dynamic activity-assignment software instructions;
where, when the at least one processor executes the fatigue-based dynamic activity-assignment software instructions, for each time period of the plurality of time periods, the at least one processor is programmed to:
receive, for each user of the plurality of users, at least:
the user-specific physical condition tracking data, and
a user-specific activity data;
automatically model, for each user of the plurality of users, a user-specific injury-prone fatigue score during each time period based, at least in part, on:
the user-specific physical condition tracking data, and
the user-specific activity data;
automatically utilize an activity-assignment data model to assign the plurality of physical activities across the plurality of users for a subsequent time period to form a subsequent instance of the dynamically-updatable activity-assignment data structure based, at least in part, on:
a current instance if the dynamically-updatable activity-assignment data structure,
the user-specific injury-prone fatigue score,
the user-specific activity data,
user-specific activity-specific ability performance data for each physical activity of the plurality of physical activities,
activity characteristic data for each physical activity of the plurality of physical activities, and
an activity-specific fatigue safety data;
transmit a plurality of activity-assignment instructions to a respective plurality of computing devices associated with the plurality of users; and
where each activity-assignment instruction of the plurality of activity-assignment instructions direct each user of the plurality of users to perform at least one particular physical activity of the plurality of physical activity during the subsequent time period at the at least one physical location of the plurality of physical locations, based, at least in part, on the subsequent instance of the dynamically-updatable activity-assignment data structure so as to reduce a likelihood of at least one fatigue-caused injury across the plurality of users performing the plurality of physical activities at the plurality of physical locations during the plurality of time periods.

2. The system of claim 1, further including:
at least one environmental condition tracking device, associated with the at least one physical location of the plurality of physical locations;
where the at least one environmental condition tracking device is configured to generate environmental condition data for at least one environmental condition metric; and
where, for each time period of the plurality of time periods, the at least one processor is further programmed to:
receive, for each user of the plurality of users, the environmental condition data and
automatically model, for each user of the plurality of users, the user-specific injury-prone fatigue score during each time period based further on the environmental condition data.

3. The system of claim 2, where the at least one environmental condition metric is one of a temperature, a humidity level, or a noise level.

4. The system of claim 1, where the activity-assignment data model is a data model associating a plurality of data definitions, including:
an activity data definition, identifying each physical activity and a performance complexity of each physical activity so as to form the activity characteristic data,
a user activity performance data definition, identifying the at least one physical activity of the plurality of physical activities that each user is capable of performing so as to form the user-specific activity-specific ability performance data, and
an activity-specific fatigue safety score data definition, identifying a ranking of the plurality of physical activities among each other from the safest to least safe to be performed when being fatigued so as to form the activity-specific fatigue safety data.

5. The system of claim 4,
where the activity-specific fatigue safety score data definition is further based, at least in part, on the plurality of physical locations so as to form location-specific activity-specific fatigue safety data; and
where, for each time period of the plurality of time periods, the at least one processor is further programmed to automatically utilize the activity-assignment data model to assign the plurality of physical activities across the plurality of users for the subsequent time period to form the subsequent instance of the dynamically-updatable activity-assignment data structure based further on the location-specific activity-specific fatigue safety data.

6. The system of claim 4, where the activity-assignment data model is further defined to assign the plurality of physical activities across the plurality of users, by:
iteratively identifying, in each iteration, the most fatigue user of the plurality of users;
iteratively identifying, in each iteration, a set of physical activities of the plurality of physical activities that the most fatigue user is capable of performing;
iteratively identifying, in each iteration, the safest physical activity in the set of physical activities;
iteratively checking, in each iteration, for a presence of another user of the plurality of users who is capable of performing a less safe physical activity of the set of physical activities;
iteratively assigning, in each iteration, the safest physical activity in the set of physical activities to the most fatigue user when the another user of the plurality of users who is capable of performing the less safe physical activity of the set of physical activities is present; and
iteratively assigning, in each iteration, the less safe physical activity in the set of physical activities to the most fatigue user under at least one activity-limiting condition when the another user of the plurality of users who is capable of performing the less safe physical activity of the set of physical activities is not present.

7. The system of claim 6, where the at least one activity-limiting condition limits a number of repetitions of the less safe physical activity that the most fatigue user is allowed to perform within the subsequent time period.

8. The system of claim 1, where, for each time period of the plurality of time periods, the at least one processor is further programmed to automatically utilize the activity-assignment data model to assign the plurality of physical activities across the plurality of users for the subsequent time period to form the subsequent instance of the dynamically-updatable activity-assignment data structure based further on user-specific historical fatigue data across a set of time periods.

9. The system of claim 8, where the set of time periods is equal to or exceeds twenty-four (24) hours.

10. The system of claim 1, where each physical activity of the plurality of physical activities is defined based, at least in part, on each job function of a plurality of job functions associated with the plurality of users.

11. The system of claim 1, where the plurality of physical locations is within a warehouse.

12. The system of claim 1, further including:
at least one camera-based tracking device, associated with the at least one physical location of the plurality of physical locations;
where the at least one camera-based tracking device is configured to generate visual tracking data for the at least one physical location; and
where, for each time period of the plurality of time periods, the at least one processor is further programmed to:
receive the visual tracking data,
utilize at least one image recognition model to recognize user-specific image data of the at least one user, and
automatically model, for each user of the plurality of users, the user-specific injury-prone fatigue score during each time period based further on the user-specific image data.

13. The system of claim 1, where the user-specific activity data includes user-specific historical fatigue data representative of past fatigue.

14. The system of claim 1, where the user-specific activity data includes user-specific input data, where the user-specific input data includes hours slept data representative of an amount of time that at least one user of the plurality of users slept during a previous night.

15. The system of claim 1, where the user-specific input data includes a subjective level of fatigue representative of a user input fatigue level of at least one user of the plurality of users.

16. A method comprising:
receiving, by a device, for each user of the plurality of users, at least:
the user-specific physical condition tracking data, and
a user-specific activity data;
automatically modelling, by the device, for each user of the plurality of users, a user-specific injury-prone fatigue score during each time period based, at least in part, on:
the user-specific physical condition tracking data, and
the user-specific activity data;
automatically utilizing, by the device, an activity-assignment data model to assign the plurality of physical activities across the plurality of users for a subsequent time period to form a subsequent instance of the dynamically-updatable activity-assignment data structure based, at least in part, on:
a current instance if the dynamically-updatable activity-assignment data structure,
the user-specific injury-prone fatigue score,
user-specific activity data,
user-specific activity-specific ability performance data for each physical activity of the plurality of physical activities,
activity characteristic data for each physical activity of the plurality of physical activities, and
an activity-specific fatigue safety data; and
transmitting, by the device, a plurality of activity-assignment instructions to a respective plurality of computing devices associated with the plurality of users,
where each activity-assignment instruction of the plurality of activity-assignment instructions direct each user of the plurality of users to perform at least one particular physical activity of the plurality of physical activity during the subsequent time period at the at least one physical location of the plurality of physical locations, based, at least in part, on the subsequent instance of the dynamically-updatable activity-assignment data structure so as to reduce a likelihood of at least one fatigue-caused injury across the plurality of users performing the plurality of physical activities at the plurality of physical locations during the plurality of time periods.

17. The method of claim 16, further comprising:
receiving, for each user of the plurality of users, the environmental condition data; and
automatically modelling, for each user of the plurality of users, the user-specific injury-prone fatigue score during each time period based further on the environmental condition data.

18. The method of claim 16, where the activity-assignment data model is a data model associating a plurality of data definitions, including:
an activity data definition, identifying each physical activity and a performance complexity of each physical activity so as to form the activity characteristic data,
a user activity performance data definition, identifying the at least one physical activity of the plurality of physical activities that each user is capable of performing so as to form the user-specific activity-specific ability performance data, and
an activity-specific fatigue safety score data definition, identifying a ranking of the plurality of physical activities among each other from the safest to least safe to be performed when being fatigued so as to form the activity-specific fatigue safety data.

19. The method of claim 16,
where the activity-specific fatigue safety score data definition is further based, at least in part, on the plurality of physical locations so as to form location-specific activity-specific fatigue safety data; and
where, for each time period of the plurality of time periods, the at least one processor is further programmed to automatically utilize the activity-assignment data model to assign the plurality of physical activities across the plurality of users for the subsequent time period to form the subsequent instance of the dynamically-updatable activity-assignment data structure based further on the location-specific activity-specific fatigue safety data.

20. The method of claim 16, where the activity-assignment data model is further defined to assign the plurality of physical activities across the plurality of users, by:
iteratively identifying, in each iteration, the most fatigue user of the plurality of users;
iteratively identifying, in each iteration, a set of physical activities of the plurality of physical activities that the most fatigue user is capable of performing;
iteratively identifying, in each iteration, the safest physical activity in the set of physical activities;
iteratively checking, in each iteration, for a presence of another user of the plurality of users who is capable of performing a less safe physical activity of the set of physical activities;
iteratively assigning, in each iteration, the safest physical activity in the set of physical activities to the most fatigue user when the other user of the plurality of users who is capable of performing the less safe physical activity of the set of physical activities is present; and
iteratively assigning, in each iteration, the less safe physical activity in the set of physical activities to the most fatigue user under at least one activity-limiting condition when the other user of the plurality of users who is capable of performing the less safe physical activity of the set of physical activities is not present
